(19) United States
(12) Patent Application Publication
(10) Pub. No.: US 2016/0242893 A1
(43) Pub. Date: Aug. 25, 2016

(54) Stent And Filter

(71) Applicant: MicroVention, Inc., Tustin, CA (US)

(72) Inventors: Kaushik Joshi, Tustin, CA (US); Matthew J. Fitz, Vista, CA (US)

(73) Assignee: MicroVention, Inc., Tustin, CA (US)

(21) Appl. No.: 15/053,970

(22) Filed: Feb. 25, 2016

Related U.S. Application Data
(60) Provisional application No. 62/120,814, filed on Feb. 25, 2015.

(51) Int. Cl. A61F 2/01

(52) U.S. Cl. CPC A61F 2/013; A61F 2002/016; A61F 2002/011

(57) ABSTRACT

A stent utilizing an integral filter is described. The filter portion of the stent can be connected directly to the end of the stent or can be space apart via a plurality of struts. The filter stent can be used to catch embolic material and/or the filter portion can be filled with embolic material (e.g., embolic coils) for the purpose of occluding a vessel.

### RELATED APPLICATIONS

This application claims priority to U.S. Provisional Application Ser. No. 62/120,814 filed Feb. 25, 2015 entitled Stent and Filter, which is hereby incorporated herein by reference in its entirety.

### BACKGROUND OF THE INVENTION

Many interventional procedures utilize a stent for various therapeutic purposes, such as to open a vessel blocked by thrombus, disease, or calcification. In many of these procedures a filter or embolic protection device is initially introduced to prevent particles from traveling through the patient's vascular system and causing complications, such as a stroke. A stent is advanced to a location proximal and upstream to the filter so that any dislodged material (e.g., thrombus) is captured by the filter and safely withdrawn from the patient. It should be noted that the filter and the stent must be delivered via separate devices and therefore the initial positioning of the filter otherwise lengthens and complicates the stent deliver procedure.

### SUMMARY OF THE INVENTION

In one embodiment a stent with a filter is described.

In one embodiment a stent with a filter and a connecting structure is described.

In one embodiment a stent with flared ends and a filter is described.

In one embodiment a stent with flared ends, a filter, and a connecting structure is described.

In one embodiment a stent with a filter is used to treat a vascular condition, and the filter is used to catch thrombus dislodged by the stent.

In one embodiment a stent with a filter is used to treat a vascular condition, and the filter is used to hold occlusive embolic material.

A stent with an integrated filter may eliminate the need for a separate filter, making the interventional procedure easier since there is no need to deploy and retrieve a separate filter.

The integrated filter portion of the stent may also be used for other purposes, such as to occlude a vessel. For example the filter may be filled with embolic coils or other embolic agents that block blood flow beyond the filter portion, occluding the vessel.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIGS. 1-2 illustrate a stent.

FIGS. 3A, 3B, and 4 illustrate a stent with flared loops.

FIGS. 5-7, 8A and 8B, and 9-16 illustrate various embodiments of stents with an integral filter.

FIG. 17A-17B illustrate a mandrel used to form a stent with an integral filter.

FIG. 18 illustrates a delivery device capable of delivering any of the filter stent embodiments of the present invention.

FIG. 19 illustrates an embolic delivery device delivering embolic coils to the filter portion of a filter stent of the present invention.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

US Pub. No. 2013/0245745 A1 describes multiple stent embodiments, including single layer and dual layer stents, and is hereby incorporated by reference in its entirety.

In various stenting procedures filters (or embolic protection devices) are first deployed at a distal location to catch thrombus dislodged when the stent is in place. A stent is subsequently introduced and expanded at a location proximal to the filter (preferably proximal and upstream of the filter), allowing the filter to catch any thrombus dislodged by the stent. This procedure is useful, for example, to open up an artery when there is thrombus formation in the artery. The following embodiments disclose a stent with an integral filter which would eliminate the need of a separate filter.

The filter basket described in the following embodiments may be comprised of a nitinol wire mesh comprising one or more braided wires. The filter may utilize radiopaque material for ease of visualization. In one example one or more of the filter mesh wires utilize a radiopaque wire (i.e. tantalum). In another example radiopaque marker bands or marker coils are placed in predetermined locations in the filter mesh. In another example the struts of the filter basket are comprised of a radiopaque material. In another example the struts of the filter basket utilize radiopaque marker bands or marker coils.

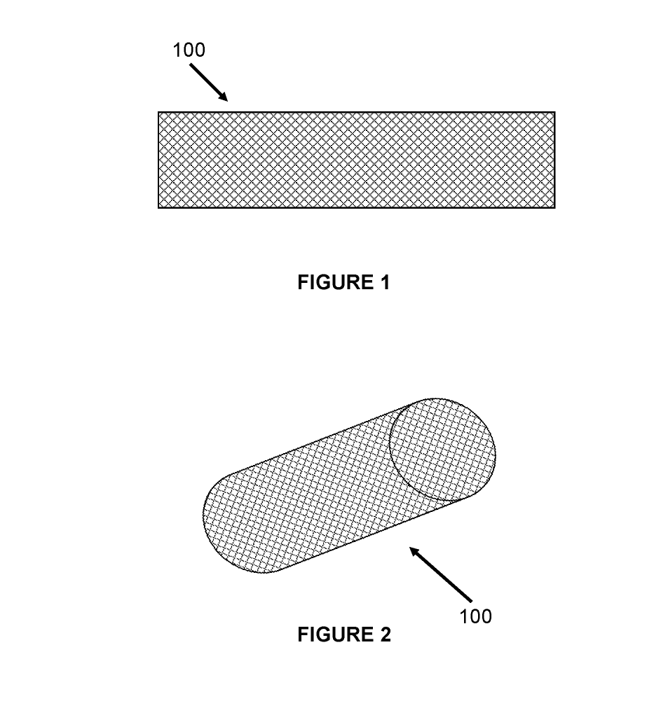

The stent 100 shown in FIGS. 1-2 is a single layer stent. In one example the stent is a self-expanding, one-layer stent comprised of a single filament wound into braided pattern. In another example the stent is a single layer comprised of a mesh of different wires interwoven together.

In another example, the stent is a self-expanding dual layer stent 200 with an inner layer or component 202 and an outer layer or component 100, see U.S. Pub. No. 2013/0245745 A1, which is incorporated above. Preferably, the inner layer 202 has a lower porosity than the outer layer 100 (i.e. outer layer has larger gap or whole sizes). The outer component can have flared distal ends at both ends of the stent. In one example both the inner layer and outer layer are comprised of a mesh of multiple wires. In another example the inner layer is a mesh of multiple wires while the outer layer is comprised of one wire wound upon itself. These flared ends provide an anchor point within the vessel thus securing the stent within the blood vessel and preventing migration.

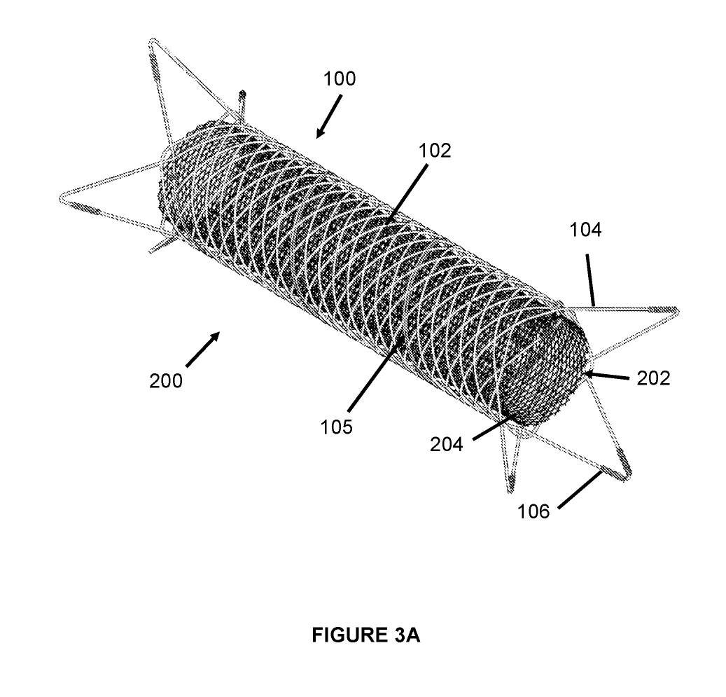

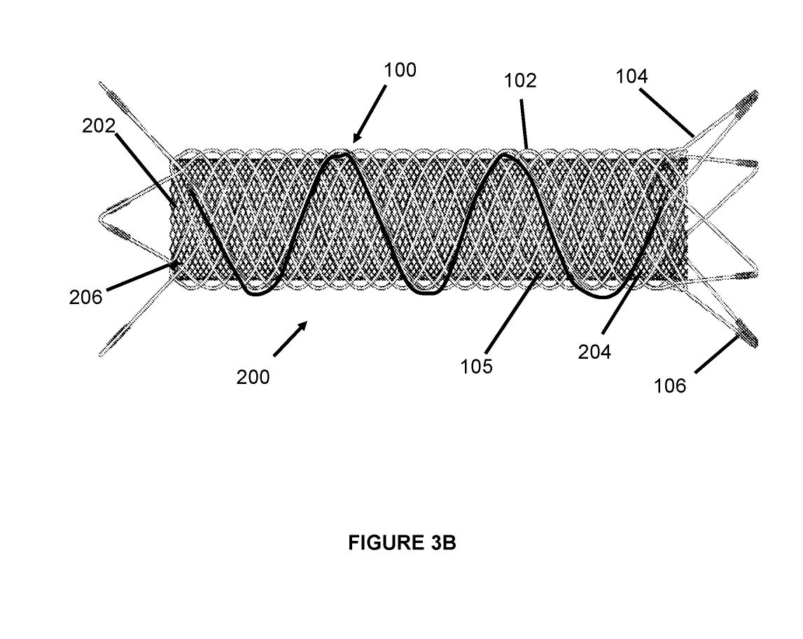

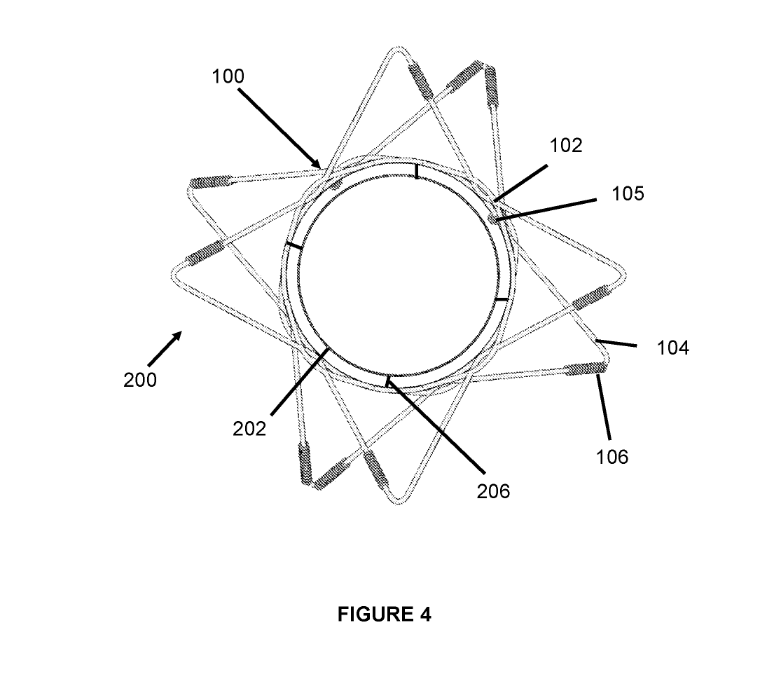

FIGS. 3A, 3B, and 4 illustrate a dual layer stent 200 comprising an inner layer 202 and outer layer 100. The inner layer 202 is composed of wires 204 woven or braided into a mesh tube shape, and the outer layer 100 is composed of one or more wires 102 woven or braided into a tube shape. The outer layer 100 includes proximal and distal radially-flared loops 104 that may include one or more radiopaque coils 106 (e.g., radiopaque wire coiled around the wire of the loop 104) used for visualization. A tantalum wire 105 may be interwoven between the inner and outer layers to bind the layers together and to promote visualization. One or more attachment members 206 (e.g., coils) may also be connected in various locations throughout the stent 200 (e.g., locations along the length of the stent) to connect the inner and outer layers of the stent. Please note the pairs of flared loops 104 shown in FIG. 4. In one example one or more of the radially, oppositely located “pair” of loops 104 is shorter than an adjacent “pair”.

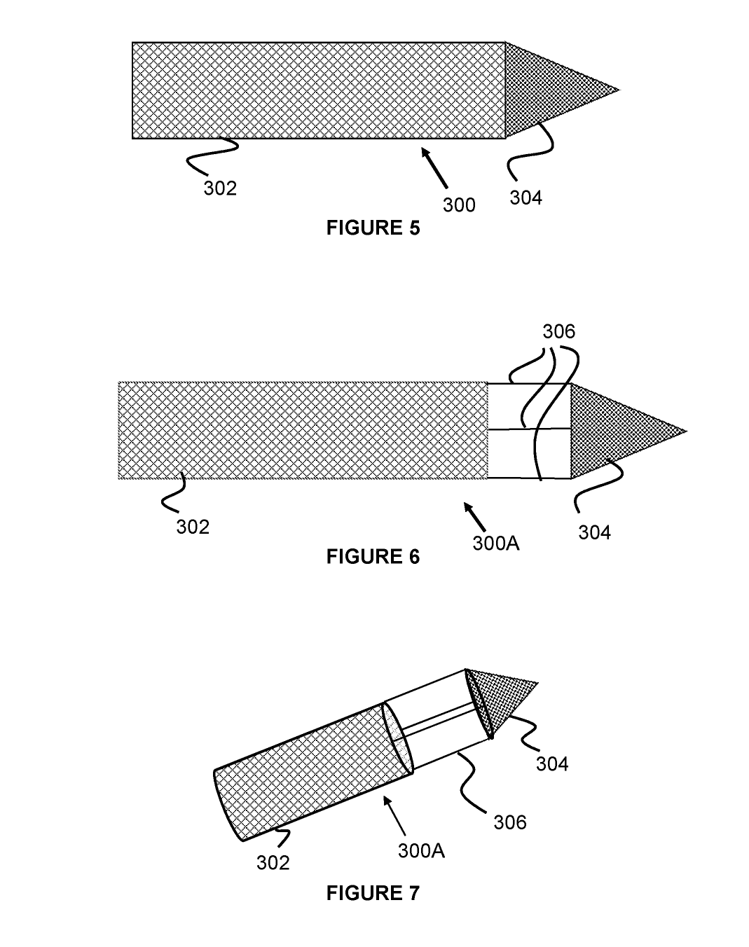

FIGS. 5-8 show an embodiment of a stent 300 composed of a stent portion 302 and a filter portion 304. The stent portion 302 can be the stent 100 shown FIGS. 1-2 or the dual layer stent 200 shown in FIGS. 3A, 3B, and 4. The filter portion 304 (also referred to as a mesh or filter basket) may be a generally concave shape, a conical shape, a bulbous, a rounded shape, a cylinder shape (with a mesh end), or a similar shape that is closed so as to block thrombus moving distally from a delivery catheter and stent.

In one example the stent portion 302 and filter portion 304 are two separately produced components which are subsequently attached to each other. The filter portion 304 can be directly welded or adhesively bonded to one end of the stent 300 (e.g., the distal end). Thus the wire or wires comprising the stent portion 302 at one end are connected to the wire or wires comprising the filter portion 304 (e.g., at intersecting points). Alternately, the filter portion 304 can be mechanically attached to one end of the stent portion 302 via mechanical ties (e.g., wires tied around wires from both portions) or coils which are tied to or wound around the intersection of, respectively, the stent portion wire/wires and the filter portion wires.

In another example the stent portion 302 and filter portion 304 are part of one commonly manufactured component. For example, the stent with integral filter 300 is wound over a mandrel in which the mandrel has a cylindrical and a conically shaped portion (or whatever filter portion shape is desired). One or more wires are wound over the cylindrical part of the mandrel to form the tubular stent portion 302, and one or more wires are wound over the conical portion of the mandrel to make up the filter portion 304. In this way, the filter portion 302 and stent portion 304 are comprised of the same wires, just wound over differently shaped portions of the mandrel to produce the respective shapes.

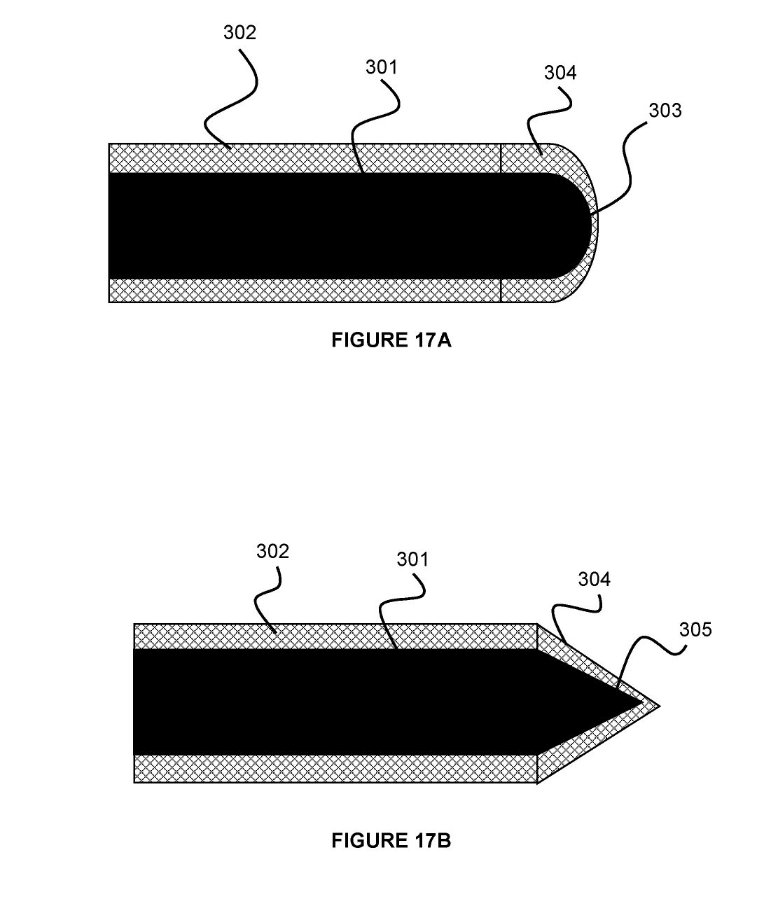

Again, the filter portion 304 is not limited to a conical shape. In one example it is a hemispherical shape in which a hemispherical-shaped mandrel section 303 is used to braid the filter portion 304 and the cylindrical mandrel portion 301 is used to braid the stent portion 302, as shown in FIG. 17A. FIG. 17B illustrates a conically-shaped mandrel portion 305 on which the conically-shaped filter portion 304 is woven.

FIGS. 6 and 7 illustrate another embodiment of a stent 300A comprised of a stent element 302, a filter element 304, and a plurality struts 306 (e.g., 4) which connect the stent portion 302 and the filter portion 304 together. In one example, the struts 306 are formed from wires braided or woven into the filter portion 304 and which are woven or connected (e.g. via welding, mechanical attachments, or adhesive). In another example, the struts 306 are part of the stent portion 302 and are wound or woven with the stent wires and connected to the filter portion 304 at its open end. This arrangement is possible where the stent portion 302 is wound and then several of the wires are cut to produce an open end of the stent portion 302, allowing the wires which are not cut to become the struts 302. The struts 302 are connect to the filter portion 304 via welding, mechanical attachment, or adhesive. In another example the struts 306 are separate from both the filter portion 304 and stent portion 302, and are instead comprised of separate wires spanning the gap and being affixed to the wires of the two portions. In another example, only some of the wires at an end of the stent portion 302 form the plurality of struts 306, leaving several uncut wires to form the struts 306. These wires forming the struts 306 can then form part of the mesh of wires comprising filter portion 304. Finally, the wires forming either the outer stent layer or inner stent layer can form the struts 306 and can continue into the filter portion 304 so as to be woven or braided into the filter portion 304.

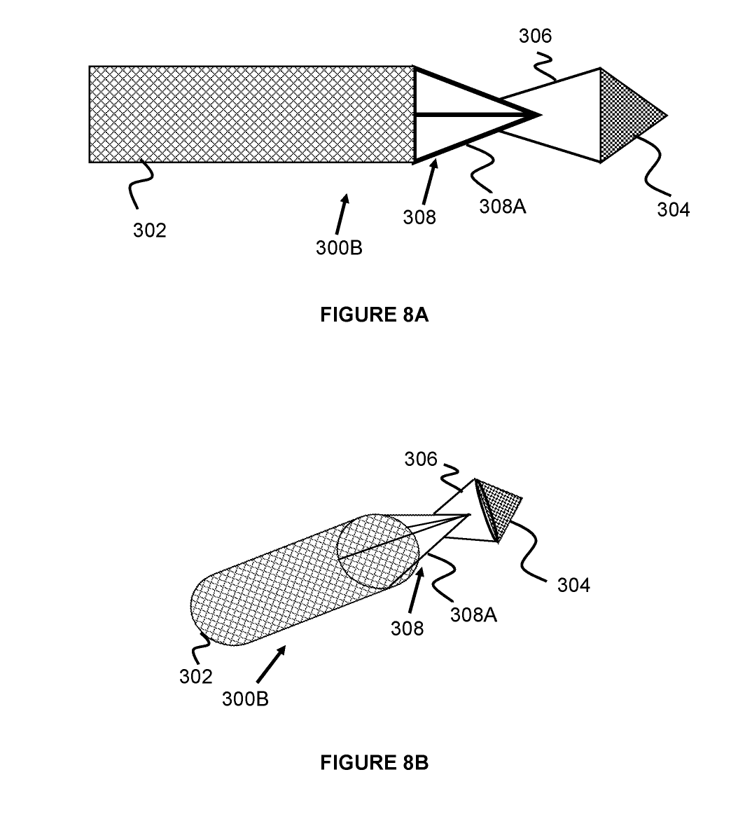

FIGS. 8A-8B illustrates another embodiment of a stent 300B comprising a stent portion 302, connecting element 308, a plurality of struts 306, and a filter portion 304. The connecting element 308 is comprised of at least two distally converging legs 308A and preferably at least four distally converting legs 308A as shown in FIGS. 8A and 8B. In one example, each of the legs 308A of connecting element 308 is comprised of two or more wires which are connected to the stent portion 302 at one end and converge a common point at the end opposite the stent portion 302. The plurality of struts 306 can connect between locations on each of the legs 308A and the filter portion 304.

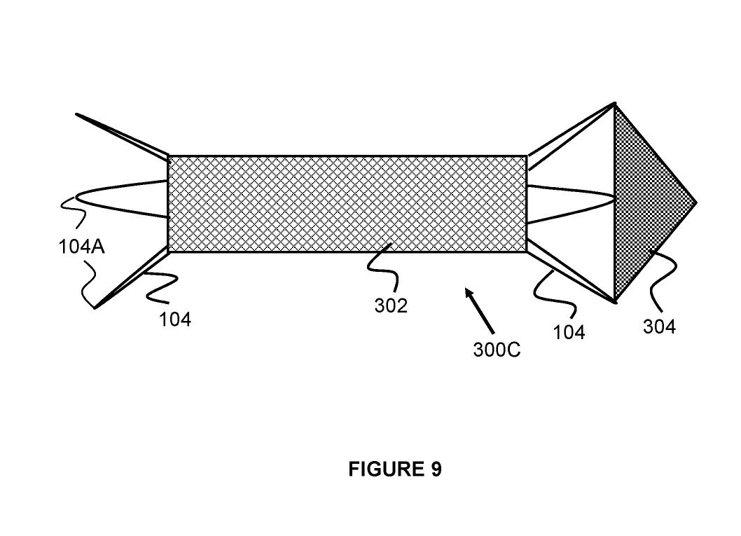

FIG. 9 illustrates another embodiment of a stent 300C having a stent portion 302 similar to the previously described dual layer stent 200 of FIGS. 3A-4, including the flared loops 104 located at both proximal and distal ends of the stent portion 302. A filter portion 304 is attached to the end of the flared loops 104. One end of the wire forming the filter portion 304 may be attached to the apex 104A of the ‘V’ shape of the flare. In embodiments with longer and shorter loops 104 at each end, the wires of the filter portion 304 may be attached to only the apexes of the longer loops 104. Alternately, the wires of the filter portion 304 may be attached to the apexes 104A of both the longer and shorter loops 104. Adhesive bonding, mechanical attachment, or welding may be used to attach the wires of filter portion 304 to the flared loops 104.

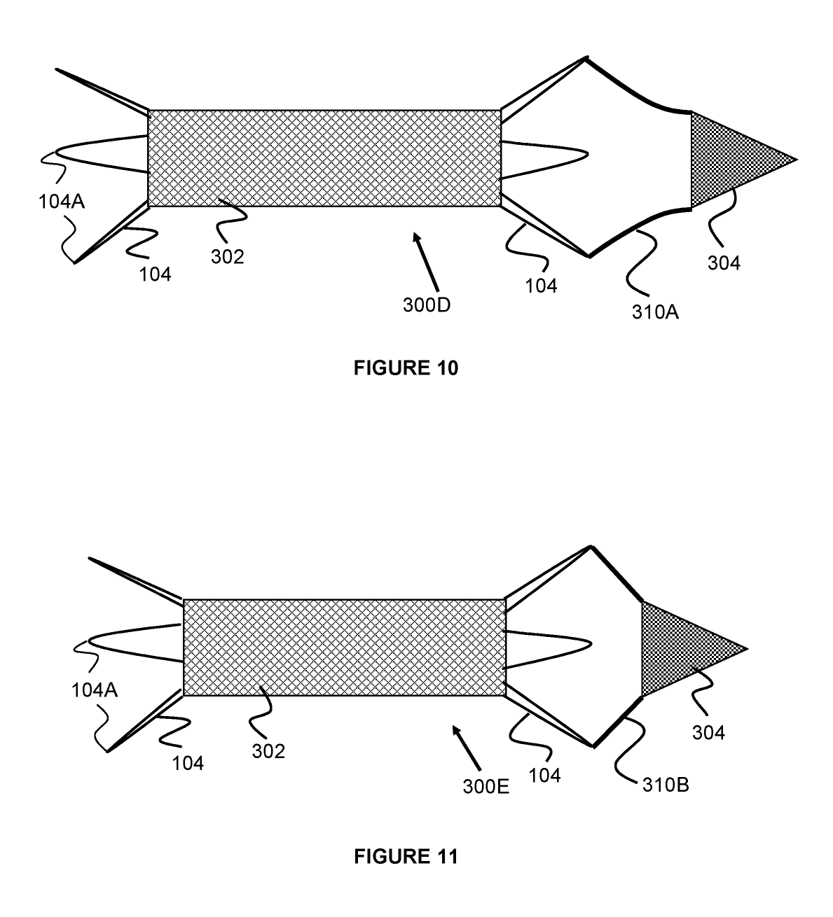

FIGS. 10-11 are variations of the embodiment of FIG. 9 in which the filter portion 304 is connected to the apex 104A of the loops 104 via connecting elements 310, which are elongated struts similar to those described in earlier embodiments. In the embodiment of the stent 300D, the connecting elements 310A are inwardly curved towards a center axis of the device, while the stent 300E includes substantially straight connector elements 310B. Preferably, at least two connecting elements 310 are used to connect to the filter portion 304 (e.g., 2, 3, 4, 5, or 6 elements 310).

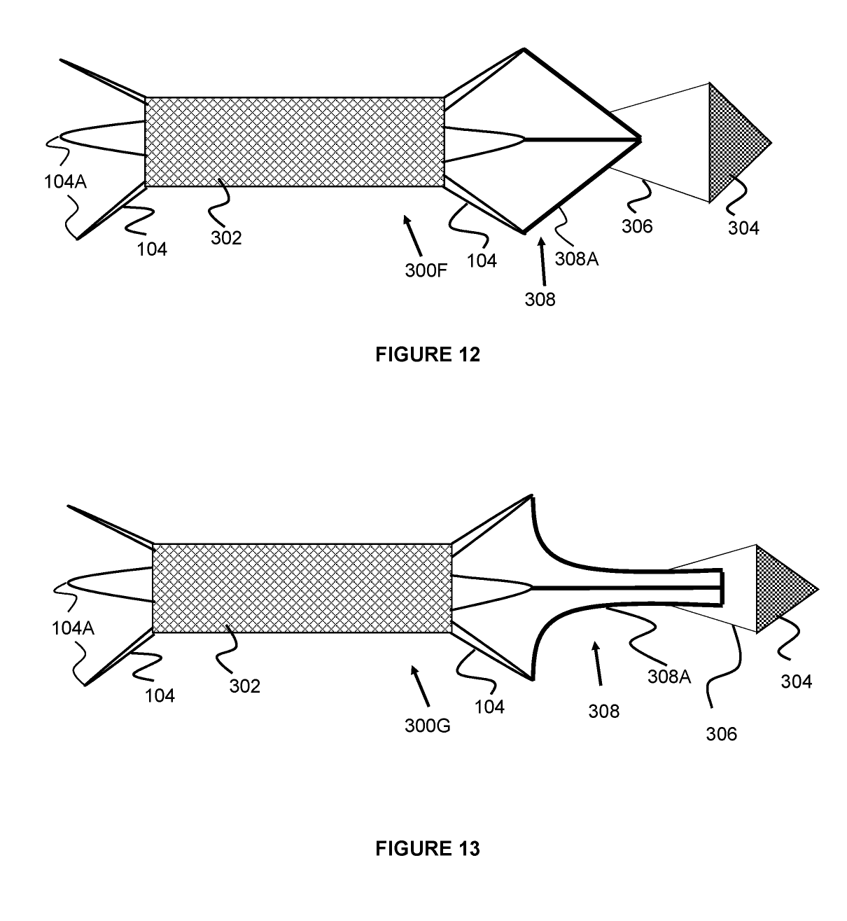

FIG. 12 illustrates another embodiment of a stent 300F having a connecting element 308 composed of a plurality of elongated leg members 308A that connects between the apexes 104A of the loops 104 and struts 306. The stent 300G of FIG. 13 is generally similar to stent 300F, except that the legs 308A of the connecting member 308 are inwardly curved towards a center axis of the stent 300G.

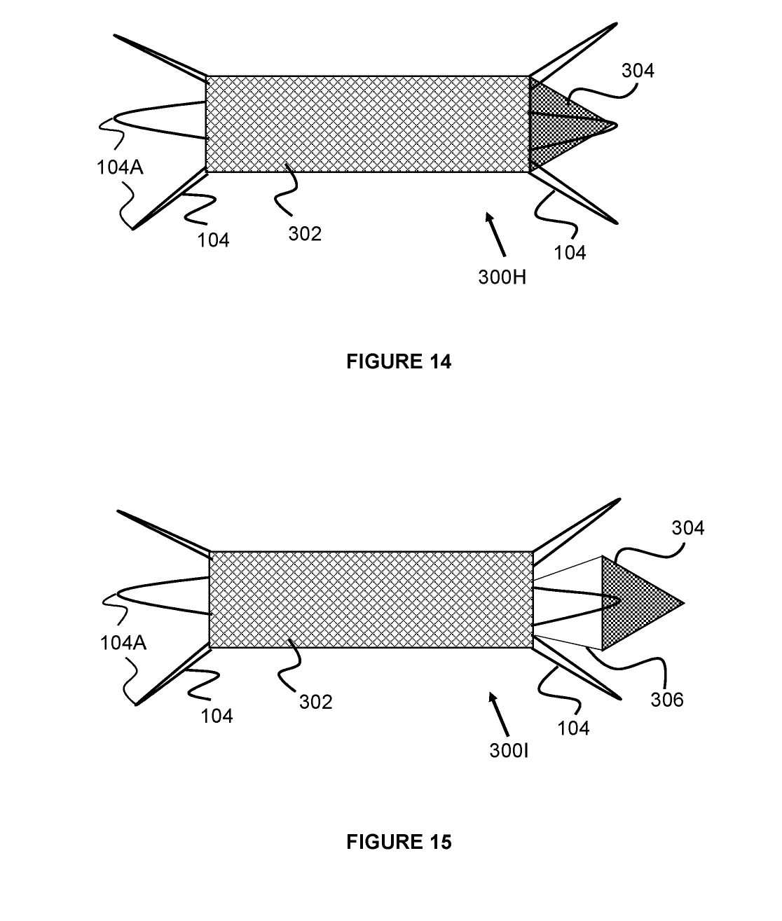

FIGS. 14 and 15 illustrate stent embodiments (300H and 300I, respectively) similar to those shown in FIGS. 5-6, except that the stent 300H or 300I is a dual layer stent with flared loops 104, as previously described in this specification. The filter portion 304 is either connected directly to the stent portion 302 or to the stent portion 302 via two or more struts 306, allowing the filter portion 304 to expand to a diameter similar to that of the stent portion 302, as opposed to the larger radius of the flared loops 104. The filter portion 304 may be attached to either the inner layer 202 or the outer layer 100 of the stent portion 302. In one example, filter portion 304 may be integrally wound with the inner layer 202 of the stent portion 302 in the manner shown and described earlier with regard to FIGS. 17A-17B.

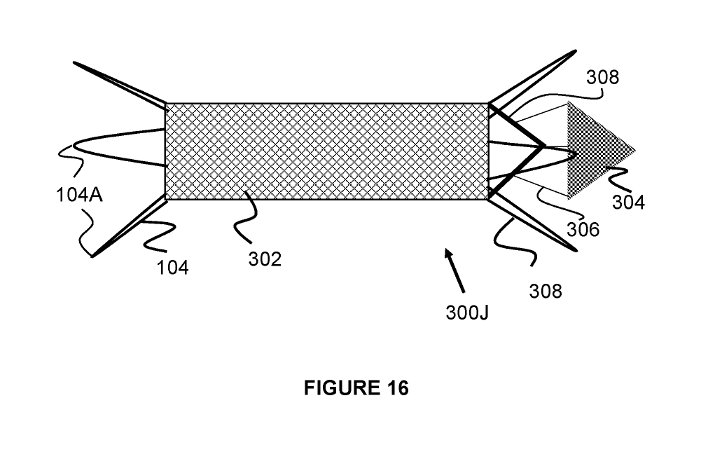

FIG. 16 illustrates an embodiment of a stent 300J utilizing the dual layer stent 200 of FIGS. 3-4 for the stent portion 302. A connecting structure 308 connects to either the inner layer 202 or the outer layer 100 of the stent 300J, and not to the loops 104 as in the embodiment of FIGS. 12-13. Struts 306 link the connecting structure 308 to the filter portion 304. As with previous embodiments, the connecting structure 308 can take on any number of shapes, such as a conical shape formed from a plurality of leg members. This embodiment is thus similar to that shown in FIGS. 8A-8B, except that the stent 300J includes an outer stent layer 100 with flared loops 104.

The stent embodiments described herein and shown in FIGS. 1-16 can be used with embolic coils that are placed within the filter portion to cause occlusion through the stent portion. For example, FIG. 19 illustrates an embolic coil delivery device 162 that has delivered one or more embolic coils 160 (and/or any other embolic material) within the filter portion 304 of any of the stent filter embodiments 300 disclosed in this specification. The embolic coil delivery device 162 can be advanced through a pre-placed catheter or sheath used by the filter stent delivery device 150, or can be separately advanced over a guidewire to a location within the deployed filter stent 300.

This arrangement may be used to close down a vessel by introducing the stent to the target vessel location and delivering the coil within in the filter portion, thereby blocking or occluding the blood flow through the blood vessel. This technique may also be useful to treat an aneurysm or other vessel malformation by positioning a stent into the aneurysm or malformation and delivering the embolic coils within the filter portion at the distal end of the filter stent in order to occlude flow beyond the filter portion and into the aneurysm or malformation. The procedure described may be used for occluding any one of: an aneurysm, left atrial appendage, fallopian tube, patent ductus arteriosus, or other occlusive procedures. In one example the stent and filter are pushed from the catheter or the sheath of the catheter is retracted to expose the stent/filter. Embolic coils are then introduced through the catheter and deployed into the filter basket.

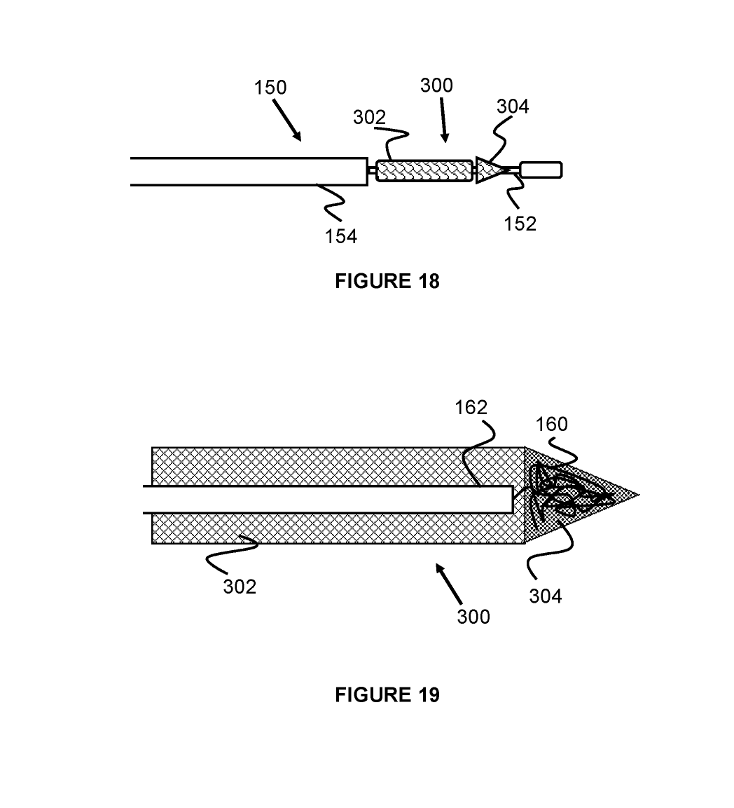

FIG. 18 illustrates one example embodiment of a delivery device 150 capable of delivering any of the filter stents 300 of the present invention. The delivery device 150 includes an inner, elongated core member 152 on which the filter stent 300 is compressed or disposed over. An outer sheath 154 is positioned over the filter stent 300 during positioning so as to maintain the filter stent 300 in its compressed configuration. Once the target location has been reached, the outer sheath 154 can be retracted by the physician, allowing the filter stent 300 to expand. The delivery device 150 may include a guidewire passage so that it alone can be advanced over the guidewire to the target location. Alternately, the delivery device can be advanced through a larger catheter or sheath that is already positioned at the target location.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A stent comprising:
a stent portion composed of one or more wires braided into a cylindrical shape; said stent portion having a proximal end and a distal end;
a filter portion connected to said distal end of said stent portion; said filter portion being comprised of mesh forming a concave shape oriented to capture material moving downstream of said stent portion.

2. The stent of claim 1, further comprising a plurality of elongated struts, each of which connected to said filter portion and to said stent portion; wherein said struts position said stent portion at a location spaced apart and distal to said distal end of said stent portion.

3. The stent of claim 2, wherein said stent portion comprises a plurality of loops that radially flare away from said stent portion and that are disconnected from said strut.

4. The stent of claim 1, further comprising:
a connecting element comprising a plurality of elongated leg members; said leg members having first ends connecting to said distal end of said stent portion and second ends converging together distally of said first ends; and,
a plurality of struts that are each connected to one of said plurality of elongated leg members and to an outer circumference of said filter portion.

5. The stent of claim 1, wherein said stent portion further comprises a plurality of loops extending from said distal end of said stent portion and that radially flare away from said stent portion; and wherein said filter portion connects to an apex of each of said plurality of loops.

6. The stent of claim 1, wherein said stent portion further comprises a plurality of loops extending from said distal end of said stent portion and that radially flare away from said stent portion; and further comprising a plurality of elongated struts, each of which connected to said filter portion and to an apex of each of said plurality of loops; wherein said struts position said stent portion at a location spaced apart and distal to said distal end of said stent portion.

7. The stent of claim 1, further comprising:
a plurality of loops extending from said distal end of said stent portion and that radially flare away from said stent portion;
a connecting element comprising a plurality of elongated leg members; said leg members having first ends connecting to an apex of each of said plurality of loops and second ends converging together distally of said first ends; and,
a plurality of struts that are each connected to one of said plurality of elongated leg members and to an outer circumference of said filter portion.

8. The stent of claim 1, further comprising:
a plurality of loops extending from said distal end of said stent portion and that radially flare away from said stent portion;
a connecting element comprising a plurality of elongated leg members; said leg members having first ends connecting to said distal end of said stent portion and second ends converging together distally of said first ends; and,
a plurality of struts that are each connected to one of said plurality of elongated leg members and to an outer circumference of said filter portion.

9. The stent of claim 1, wherein said stent portion comprises an outer cylindrical layer having a first porosity and an inner cylindrical layer having a second porosity that is less than said first porosity.

10. The stent of claim 1, wherein said one or more wires of said stent portion are further braided so as to form said filter portion.

11. The stent of claim 2, wherein said one or more wires of said stent portion extend from said distal end of said stent portion so as to form said plurality of elongated struts.

12. The stent of claim 2, wherein said one or more wires of said stent portion extend from said distal end of said stent portion so as to form said plurality of elongated leg members.

13. The stent of claim 1, wherein said stent portion comprises an outer cylindrical layer woven from outer wires and having a first porosity, and an inner cylindrical layer woven from inner wires and having a second porosity that is less than said first porosity; and wherein said filter portion is connected to and woven from said inner wires of said inner cylindrical layer.

14. The stent of claim 1, further comprising a plurality of elongated struts, each of which connected to said filter portion and to said inner cylindrical layer of said stent portion; wherein said struts position said stent portion at a location spaced apart and distal to said distal end of said stent portion and wherein said struts are formed from said inner wires of said inner cylindrical layer.

15. The stent of claim 4, wherein said plurality of leg members are curved inwards towards a central axis of said stent.

16. A method of occluding a vessel, comprising:
deploying a stent comprising:
a stent portion composed of one or more wires braided into a cylindrical shape;
said stent portion having a proximal end and a distal end;
a filter portion connected to said distal end of said stent portion; said filter portion being comprised of mesh forming a concave shape oriented to capture material moving downstream of said stent portion;
advancing an embolic delivery device to said stent; and,
delivering embolic coils within said filter portion.

17. The method of claim 16, wherein said deploying said stent further comprises advancing a stent delivery device to a target location; retracting an outer sheath of said delivery device so as to allow said stent to radially expand; and withdrawing said stent delivery device.
